(19) United States
(12) Patent Application Publication
(10) Pub. No.: US 2019/0183956 A1
(43) Pub. Date: Jun. 20, 2019

(54) USE OF A CLOVE OIL FRACTION

(71) Applicant: André Moreira CASTILHO, Belo Horizonte (BR)

(72) Inventor: Luiz Francisco PIANOWSKI, Bragança Paulista (BR)

(21) Appl. No.: 16/318,881

(22) PCT Filed: Jul. 17, 2017
(86) PCT No.: PCT/BR2017/050194

§ 371 Date: Jan. 18, 2019

(30) Foreign Application Priority Data
Jul. 18, 2016 (BR) 10 2016 016555 5

(51) Int. Cl. A61K 36/61; A61K 47/44; A61K 45/06; A61P 29/00

(52) U.S. Cl. CPC A61K 36/61; A61K 47/44; A61K 9/0014; A61P 29/00; A61K 45/06

(57) ABSTRACT

The present invention relates to a method and composition for treating inflammatory conditions of the animal body using a clove oil fraction, particularly a fraction that remains after removal of eugenol. Such a composition can be used as an analgesic and an anti-inflammatory.

### FIELD OF THE INVENTION

The present invention relates to a method and composition for treating inflammatory conditions of the animal body using a clove oil fraction, particularly a fraction that remains after removal of eugenol. Such a composition can be used as an analgesic and an anti-inflammatory.

### BACKGROUND OF THE INVENTION

The essential oil of clove (Syzygium aromaticum, Cariophylus arimathicus L, Eugenia aromatica (L.) Baill., Eugenia caryophyllata Thunb., Eugenia caryophyllus (Spreng.)), also commonly called clove, is known and used in folk medicine. Its main constituent is eugenol, in typical contents of 70-90%.

Eugenol is an aromatic compound named 4-allyl-2-methoxyphenol, and it is known to have anti-inflammatory, healing, antiseptic and analgesic properties. Clove oil is commonly cited as a synonym for eugenol given that it is present in high content, however, there are certain components of clove oil that are not derived from eugenol.

Eugenol is widely used in dentistry as an anesthetic for the relief of toothaches. In the process of preparing the anesthetic, eugenol is extracted from clove oil and the rest is discarded.

However, other components of clove oil have interesting characteristics, such as transcariophyllene (or beta-caryophyllene) and alpha-humulene, which are reported to be present at amounts of 5-12% and 1-1.5% of the total oil, respectively.

These transcariophyllene and alpha-humulene compounds are known to be effective in treating inflammatory conditions of the animal body, particularly inflammatory pain or inflammatory edema.

### SUMMARY OF THE INVENTION

The invention provides a composition and a method for treating inflammatory conditions of an animal body. The composition comprises a therapeutically effective amount of a clove oil fraction, which is a fraction that remains after the removal of eugenol, and an excipient. The method for treating an inflammatory condition comprises administering said composition to an animal body.

### DETAILED DESCRIPTION OF THE INVENTION

The invention seeks to provide a method and composition for treating inflammatory conditions of the animal body using a clove oil fraction, said fraction being a fraction remaining after removal of eugenol.

As used herein, “animal body” refers to a human body.

As used herein, “a fraction remaining after removal of eugenol” refers to any fraction that remains after any amount of eugenol has been removed from clove oil, or any fractions obtained therefrom, where additional components that are not transcaryophyllene or alpha-humulene have been removed.

In one embodiment of the invention, the composition containing a clove oil fraction for the treatment of inflammatory conditions of the animal body allows for the preparation of salts and derivatives of alpha-humulene and transcariophyllene.

In one embodiment of the invention, the method and composition for treating inflammatory conditions of the animal body using a clove oil fraction refers to conditions that cause inflammation, inflammatory pain and inflammatory edema.

In one embodiment of the invention, the composition containing a clove oil fraction for the treatment of inflammatory conditions of the animal body is an additive to a medicament, an adjuvant to a medicament or a medicament itself.

In some embodiments of the invention, the composition containing a clove oil fraction for the treatment of inflammatory conditions of the body is suitable for enteral or parenteral administration, including topical, transdermal, subcutaneous, intraperitoneal, intravenous, by infiltration, by inhalation, trans-mucosal, intramuscular, intrapulmonary, vaginal, rectal, intraocular and sublingual.

In some embodiments of the invention, the composition containing a clove oil fraction for the treatment of inflammatory conditions of the body may be in the form of solid, liquid, semisolids or pastes, pills, tablets, hard or soft capsules, lozenges, powders, granules, suspensions, dispersions, emulsions, and any other form known to the person skilled in the art.

In some embodiments of the invention, the composition containing a clove oil fraction may be of immediate or extended release, and may make use of micro or nanoparticles.

In some embodiments of the invention, some of the inflammatory conditions treated by the composition containing a clove oil fraction are one or more of rheumatoid arthritis, osteoarthritis, systemic lupus erythematosus, ulcerative colitis, psoriasis, atopic eczema, atherosclerosis, and non-degenerative diseases, e.g., depression, cellulitis and allergy.

The composition containing a clove oil fraction according to the invention typically contains excipients known to a person skilled in the art, for example those mentioned in the publication “Remington—The Science and Practice of Pharmacy” by Lippincott Williams & Wilkins, 2006.

In some embodiments of the invention, the composition containing a clove oil fraction for the treatment of inflammatory conditions of the body is to be administered orally and contains from 1 mg to 1000 mg of the transcariophyllene and alpha-humulene mixture as contained in the clove oil fraction upon removal of eugenol, particularly between 10 and 100 mg, and more particularly from 30 to 100 mg.

For topical use, the product contains 0.01 to 100% of the clove oil fraction.

In one embodiment of the invention, the composition containing a clove oil fraction for the treatment of inflammatory conditions of the body may also contain agents other than those contained in said fraction, e.g. one or more of an antibiotic, a muscle relaxant, hormones, an anti-hyperlipidemic, an antiallergic, vitamins, calcium sources, antidepressants, healing, etc.

One skilled in the art may, from the information provided herein, carry out the invention in equivalent forms not expressly described, but with the same or substantially the same function, or the same or substantially the same results, while still being within the scope of the appended claims.

1-9. (canceled)

10. A composition for the treatment of inflammatory conditions of an animal body comprising a therapeutically effective amount of a clove oil fraction, said fraction being a remaining fraction after removal of eugenol, and an excipient.

11. The composition according to claim 10, wherein the inflammatory conditions are selected from the group consisting of conditions that cause inflammation, conditions that cause inflammatory pain or conditions that cause inflammatory edema.

12. The composition according to claim 10, wherein the composition is used as an additive to a medicament, an adjuvant to a medicament or a medicament itself.

13. The composition according to claim 10, suitable for enteral or parenteral administration, the administration selected from the group consisting of topical, transdermal, subcutaneous, intraperitoneal, intravenous, by infiltration, by inhalation, trans-mucosal, intramuscular, intrapulmonary, vaginal, rectal, intraocular and sublingual.

14. The composition according to claim 10, prepared in a form selected from the group consisting of a solid, liquid, semisolid or paste, pill, tablet, capsule, lozenge, powder, granule, suspension, dispersion and emulsion.

15. The composition according to claim 10, wherein the inflammatory condition is selected from the group consisting of rheumatoid arthritis, osteoarthritis, systemic erythematosus lupus, ulcerative colitis, psoriasis, atopic eczema, atherosclerosis, depression, cellulitis, allergy, and other non-degenerative diseases.

16. The composition according to claim 10 wherein the composition comprises 1 mg to 1000 mg of a transcariophyllene and alpha-humulene mixture.

17. The composition according to claim 16 wherein the composition comprises 10 mg to 100 mg of a transcariophyllene and alpha-humulene mixture.

18. The composition according to claim 16 wherein the composition comprises 30 mg to 100 mg of a transcariophyllene and alpha-humulene mixture.

19. The composition according to claim 10 containing 0.01% to 100% of the clove oil fraction.

20. The composition according to claim 10, further comprising one or more agents in addition to those contained in said fraction, wherein the one or more agents is selected from the group consisting of antibiotic, muscle relaxant, hormone, antihyperlipidemic, antiallergic, vitamin, calcium source, antidepressant and healing agents.

21. A method for treating an inflammatory condition comprising administering the composition of claim 10 to an animal body.

22. The method according to claim 21, wherein the inflammatory condition is selected from the group consisting of a condition that cause inflammation, a condition that causes inflammatory pain, and a condition that causes inflammatory edema.

23. The method according to claim 21, wherein the composition is selected from the group consisting of an additive to a medicament, an adjuvant to a medicament or a medicament itself.

24. The method according to claim 21, wherein the composition is suitable for enteral or parenteral administration selected from the group consisting of topical, transdermal, subcutaneous, intraperitoneal, intravenous, by infiltration, by inhalation, trans-mucosal, intramuscular, intrapulmonary, vaginal, rectal, intraocular and sublingual administration.

25. The method according to claim 21, wherein the composition is suitable for oral administration.

26. The method according to claim 21, wherein the composition is prepared in a form selected from the group consisting of; a solid, liquid, semisolid or paste, pill, tablet, capsule, lozenge, powder, granule, suspension, dispersion and an emulsion.

27. The method according to claim 21, wherein the inflammatory condition is selected from the group consisting of rheumatoid arthritis, osteoarthritis, systemic erythematosus lupus, ulcerative colitis, psoriasis, atopic eczema, atherosclerosis, depression, cellulitis, allergy, and other non-degenerative diseases.

28. The method according to claim 21, wherein the composition comprises 1 mg to 1000 mg of a transcariophyllene and alpha-humulene mixture.

29. The method according to claim 21, wherein the composition comprises 10 mg to 100 mg of a transcariophyllene and alpha-humulene mixture.

30. The method according to claim 21, wherein the composition comprises 30 mg to 100 mg of a transcariophyllene and alpha-humulene mixture.

31. The method according to claim 21, wherein the composition contains 0.01% to 100% of the clove oil fraction, administered topically.

32. The method according to claim 21, wherein the composition further comprises one or more agents in addition to those contained in said fraction, wherein the one or more agents is selected from the group consisting of antibiotic, muscle relaxant, hormone, antihyperlipidemic, antiallergic, vitamin, calcium source, antidepressant and healing agents.
